(19) United States
(12) Patent Application Publication
(10) Pub. No.: US 2020/0172850 A1
(43) Pub. Date: Jun. 4, 2020

(54) ELECTROCHEMICAL CONTROL OF REDOX POTENTIAL IN BIOREACTORS

(71) Applicant: The Regents of the University of Colorado, a body corporate, Denver, CO (US)

(72) Inventor: Zhiyong Ren, Superior, CO (US)

(21) Appl. No.: 16/698,306

(22) Filed: Nov. 27, 2019

Related U.S. Application Data
(60) Provisional application No. 62/772,687, filed on Nov. 29, 2018.

(51) Int. Cl. C12M 1/34; C12P 7/52; C12P 7/54; C12P 7/56; C12P 5/02; C12M 1/12

(52) U.S. Cl. CPC C12M 41/28; C12P 7/52; C12M 25/02; C12P 7/56; C12P 5/023; C12P 7/54

(57) ABSTRACT

Described herein are methods for using solid electrodes as an alternative source or sink of electrons to regulate the redox potential of mixed culture anaerobic reactors, so tunable fermentation products can be generated.

### CROSS-REFERENCE TO RELATED APPLICATION

The present application claims priority under 35 U.S.C. § 119(e) to U.S. Provisional Patent Application No. 62/772,687, filed Nov. 29, 2018, which application is incorporated herein by reference in its entirety.

### BACKGROUND

Anaerobic processes play a role in transforming traditional energy intensive wastewater treatment plants towards energy and resource recovery facilities. Anaerobic digestion (AD) is a model technology that breaks down and stabilizes organic waste such as wastewater sludge, food waste, and animal waste and generates biogas and nutrient-rich effluent and bio-solids. Therefore, there is a continued need for a system that may be manipulated to produce desired metabolic byproducts from organic waste. Such a method and system would be extremely useful in producing useful byproducts, such as methane or other short chain metabolites.

### BRIEF SUMMARY OF THE INVENTION

Provided herein is a method for using solid electrodes as an alternative source or sink of electrons to regulate the redox potential of mixed culture anaerobic reactors, so tunable fermentation products can be generated. In one embodiment, a method of producing the fermentation products of mixed culture includes; a) providing; i) a mixed culture comprising at least two different types of microorganisms; ii) at least one reactor, wherein said reactor includes at least two chambers, wherein said first chamber includes at least one anode and said second chamber includes at least one cathode; and iii) a cation exchange membrane or other separator separating said first and second chambers; b) adjusting the electrical potential poised on the electrodes so as to modify the fermentation products of said mixed culture; and c) collecting said fermentation products. In one embodiment, said reactor includes an anaerobic bioreactor. In one embodiment, said ion exchange membrane includes a cation exchange membrane. In one embodiment, said modification of the fermentation products of said mixed culture is compared to the process without applied potential. In one embodiment, the range of voltage is −2.0V to +2.0V. In one embodiment, said fermentation products include short chain volatile fatty acids, alcohol, hydrogen, and methane gas. In one embodiment, a cathodic current and lower reactor working potential increases CH₄ generation. In one embodiment, said cathodic current includes lower than −0.2 V working potential with the applied electrode potential ranged from −2.0 V to +2.0 V (vs. Ag/AgCl). In one embodiment, an anodic current and higher working potential increases volatile fatty acids generation.

In one embodiment, said a working potential anodic current comprises higher than −1.0 V working potential with the applied electrode potential ranged from −2.0 V to +2.0 V (vs. Ag/AgCl). In one embodiment, a cathodic current increases CH₄ generation. In one embodiment, lower than −1.0 V working potential increases CH₄ generation. In one embodiment, said cathodic current comprises lower than −1.0 V working potential. In one embodiment, an anodic current increases volatile fatty acids generation.

In one embodiment, a reactor working potential less than the open circuit potential induces a cathodic current and increases CH₄ generation. In another embodiment, a reactor working potential greater than the open circuit potential induces an anodic current and increases short chain volatile fatty acid generation. In some embodiments, a reactor working potential greater than −0.60V induces an anodic current and increases short chain volatile fatty acid generation. In one embodiment, said applied potentials on the electrodes influence the fermentation product distribution. In one embodiment, said reactor further includes a Ag/AgCl reference electrode. In one embodiment, said reactor further includes a gas bag connected to the working electrode chamber using a collection tube. In one embodiment, said reactor further includes growth medium purged with N₂. In one embodiment, said mixed culture includes anaerobic wastewater sludge. In one embodiment, said mixed culture is selected from at least one of the group consisting of Actinobacillus succinogenes, Escherichia coli, fermentative bacteria, methanogens, acetogens, acidogenic bacteria, electroactive bacteria, and those from wastewater, sewage sludge, and other indigenous bacteria in the environment. In one embodiment, said mixed culture is selected from at least one of the group consisting of fermentative bacteria, methanogens, acetogenic bacteria, acidogenic bacteria, and electroactive bacteria. Non-limiting examples of electroactive bacteria include Clostridium spp., Geobacter spp., and/or Methanobacteraceae spp.

Also provided is a method for using solid electrodes as an alternative source or sink of electrons to regulate the redox potential of mixed culture anaerobic reactors, so tunable fermentation products can be generated. In one embodiment, the method of producing the fermentation products of mixed culture includes; a) providing; i) a mixed culture comprising at least two different types of microorganisms; ii) at least one reactor, wherein said reactor includes at least two chambers, wherein said first chamber includes an anode chamber and said second chamber includes a cathode chamber; and iii) a cation exchange membrane separating said first and second chambers; b) adjusting the electrical potential poised on the electrode so as to modify the fermentation products of said mixed culture; and c) collecting said fermentation products. In one embodiment, said reactor includes an anaerobic reactor.

In one embodiment, said fermentation products comprise short chain volatile fatty acids. In one embodiment, said reactor under −1.0 V working potential increases CH₄ generation. In one embodiment, said reactor over −1.0 V working potential reduces CH₄ generation. In one embodiment, said reactor above −0.60 V working potential increases butyric acid generation. In one embodiment, said reactor further includes a Ag/AgCl reference electrode. In one embodiment, said reactor further includes a gas bag connected to the working electrode chamber using a collection tube. In one embodiment, said reactor further includes growth medium purged with N₂. In one embodiment, said mixed culture includes anaerobic wastewater sludge. In one embodiment, at least one of said microorganism includes M. thermautotrophicus.

In one form, the first electrode includes neutral red covalently bound to graphite felt. It has been discovered that the efficiency of electron transfer from microbial cells to electrodes can be increased by covalently linking neutral red to woven graphite felt. Neutral red can be covalently linked to graphite felt by converting at least a portion of the surface of the graphite felt to its carboxy form (using heat, for example) and covalently linking the amine group of neutral red to a carboxy group in the woven graphite.

### BRIEF DESCRIPTION OF THE FIGURES

The drawings illustrate generally, by way of example, but not by way of limitation, various embodiments of the present application.

FIGS. 1A-1B show the temporal changes of working potential (FIG. 1A) and electric current (FIG. 1B) monitored for electrofermentation (EF) reactors during 93-day operation. The reactors were either controlled with different potentials (day 0-70) or in open circuit (day 71-93). The light colored arrows show substrate replacement. The initial pH of the fermentation broth was separately adjusted to 7.0 (day 0-37), and 6.2 (day 38-93).

FIGS. 2A-2B show the profiles of H₂ (FIG. 2A) and CH₄ production (FIG. 2B) in EF reactors operated in different stages. The light colored arrows indicate substrate replacement and one batch duration. The initial pH was separately adjusted to 7.0 (0-37 days), and 6.2 (38-93 days).

FIG. 3 shows the final pH of the fermentation electrolyte with three different operation conditions (initial pH 7.0 with controlled working potentials (7.0-EF), initial pH 6.2 with controlled working potentials (6.2-EF), and initial pH 6.2 with open circuit (6.2-OC). The initial pH was shown with the dashed line.

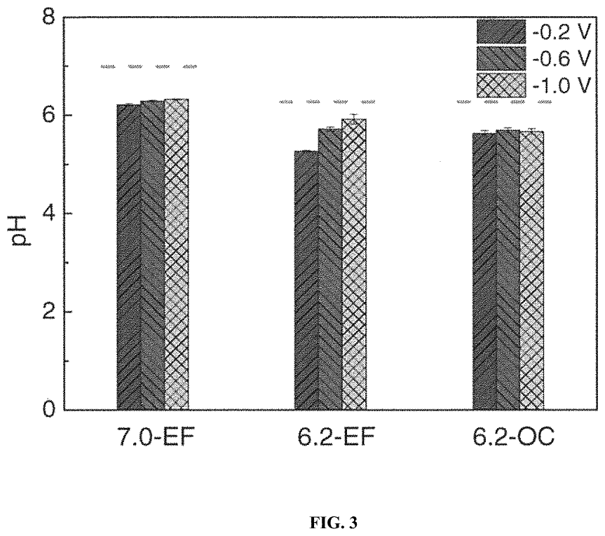

FIGS. 4A-4C show the concentration of carboxylic acids in EF reactors with different working potentials: (FIG. 4A) −0.2 V, (FIG. 4B) −0.6 V, and (FIG. 4C) −1.0 V. The initial pH was adjusted to 7.0 using 200 mM PBS.

FIGS. 5A-5C show the concentration of carboxylic acids in EF reactors with different working potentials: (FIG. 5A) −0.2 V, (FIG. 5B) −0.6 V, and (FIG. 5C) −1.0 V. The initial pH was adjusted to 6.2 using 200 mM PBS.

FIG. 6A-6C show the concentration of carboxylic acids in EF reactors under open circuit (QC) but previously controlled with different working potentials: (FIG. 6A) −0.2 V, (FIG. 6B) −0.6 V, and (FIG. 6C) −1.0 V. The initial pH was adjusted to 6.2 using 200 mM PBS.

FIGS. 7A-7D show the concentration of total VFAs (FIG. 7A), acetic acid (FIG. 7B), propionic acid (FIG. 7C) and butyric acid (FIG. 7D) in EF reactors in different stages. “7.0-EF” and “6.2-EF” indicate the initial pH in closed circuit EFs was 7.0 and 6.2, respectively. “6.2-OC” indicates open circuit condition with an initial pH of 6.2.

FIGS. 8A-8B show cyclic voltammetry (CV) profiles of the working electrodes under different poised potentials at (FIG. 8A) the beginning and (FIG. 8B) the end of the one batch cycle.

FIGS. 9A-9B show time-course cumulative CH₄ production from different sludge-substrate mixtures tailored for different methanogen groups (formate, acetate, H₂/CO₂) (FIG. 9A), and H₂ consumption profile related to hydrogenotrophic methanogenesis (FIG. 9B).

FIG. 10 shows a schematic of mixed culture glucose fermentation pathway under the influence of electrochemical control of redox potential.

FIG. 11 shows one embodiment of the mixed culture anaerobic reactors illustrating the working electrodes.

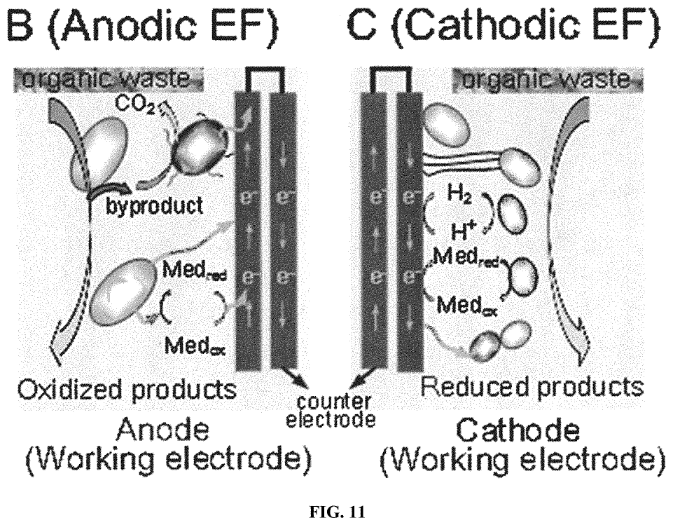

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter. One skilled in the relevant art will recognize, however, that the subject matter described herein may be practiced without one or more of the specific details, or with other methods, components, materials, and so forth. In other instances, well-known structures, materials, or operations are not shown or described in detail to avoid obscuring aspects of the subject matter herein.

References throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “anodic current” comprises greater than −1.0 V working potential.

As used herein, the term “cathodic current” comprises lower than 1.0 V working potential.

As used herein, the term “aerobic digestion” refers to a system in which gaseous oxygen is allowed to entering the system.

As used herein, the term “anaerobic digestion” refers to a collection of processes by which microorganisms break down biodegradable material in the absence of oxygen. In an anaerobic digestion system a system gaseous oxygen is prevented from entering the system through physical containment. Fermentation is one process within anaerobic digestion.

As used herein, the term “anaerobic bioreactor” refers to a system in which gaseous oxygen is prevented from entering the system through physical containment.

Various biocatalysts are suitable for use in an electrochemical bioreactor system in accordance with the methods described herein. For example, the biocatalyst can include an oxidoreductase (e.g., fumarate reductase bound to the first electrode). The biocatalyst can also include bacterial cells disposed in the first compartment of the electrochemical bioreactor system. Non-limiting examples of bacterial cells include cells of Actinobacillus succinogenes, Escherichia coli, fermentative bacteria, methanogens, acetogens, acidogenic bacteria, electroactive bacteria, and those from wastewater, sewage sludge, and other indigenous bacteria in the environment.

### Methods of Tuning Fermentation Products

Described herein are methods of using solid electrodes as an alternative source or sink of electrons to regulate the redox potential of mixed culture anaerobic reactors, so tunable fermentation products can be generated. The product spectrum was characterized under the working potentials of −1.0 V, −0.6 V, and −0.2 V, which spans the electron flow direction from cathodic current to anodic current. Results show that low working potential led to higher production of CH₄, H₂ and acetic acid, while increasing the potential from −1.0 V to −0.2 V greatly reduced methanogenesis by 68% and acetic acid generation by 58% in neutral pH. In the meantime, butyric acid production increased by 25%, while propionic acid concentration maintained stable. This redox potential based control presents a new approach to regulate the mixed culture fermentation and improve product tunability.

Without being bound by theory, it is believed that anaerobic processes play a role in transforming traditional energy intensive wastewater treatment plants towards energy and resource recovery facilities. Without being bound by theory, it is believed that anaerobic digestion (AD) is a technology that breaks down and stabilizes organic waste such as wastewater sludge, food waste, and animal waste and generates biogas, nutrient-rich effluent, and bio-solids. The AD process corresponds to a cascade of oxidation and reduction reactions carried out by consortia of microorganisms, and CH₄ is the main final product, because it has the lowest oxidation state. Without being bound by theory, it is believed that AD is a closed system without external inputs of electron acceptors or energy source, so it tends to reach a thermodynamic equilibrium with the products (CH₄) having the lowest Gibbs energy change per electron than any other organic compound during biological conversion. This high specificity can provide AD an advantage over other bioenergy systems, as it generates a homogeneous and easily separable gaseous product despite the vast heterogeneity of the substrate. However, biogas is a very low-value product, and it requires clean-up before use or it must go through significant upgrading to meet pipeline quality standards. With the abundant supply of cheap natural gas, biogas production from AD faces significant challenges in applications, as current economics cannot justify the investment in biogas. Additionally, obtaining air permits has been another major barrier due to air and greenhouse gas regulations. CH₄ is a potent greenhouse gas with a global warming potential 25-45 times higher than carbon dioxide.

New initiatives to increase the valorization of organic waste involve the production of short chain volatile fatty acids (VFAs) and alcohols via anaerobic fermentation. Such products not only bring up the values by themselves as compared to biogas, they are also useful chemical precursors for the production of even higher valued chemicals such as polyhydroxyalkanoates (PHAs), biofuels, and single cell protein (SCP). To improve the recovery of VFAs and alcohols, factors such as substrate, pH, temperature, and hydraulic retention time (HRT) are generally optimized to regulate the product spectrum. For example, lower pH values (4.5-7.0) were found favorable for mixed butyrate/acetate generation with methanogenesis inhibition, while higher pH (7.0-8.5) and thermophilic temperatures led to higher alcohol content. Similar to pH as a measure of proton activity, the extracellular ORP (oxidation reduction potential) corresponds to the activity of the electrons present in the electrolyte and can be easily monitored with an ORP sensor, which represents important operational conditions that influences the NAD+/NADH ratio within cells. The NAD+/NADH ratio represents the intracellular ORP because of intracellular redox homeostasis, which controls gene expression and enzyme synthesis for the overall cell metabolic activities. Without being bound by theory, it is believed that that by controlling the redox potential or ORP in the reactor, the fermentation pathways can be influenced and therefore the product spectrum can be regulated. It has been reported that under −0.8 V, CH₄ production and growth of M. thermautotrophicus were increased by 1.6 and 3.5 times compared with a control, while methanogenesis was effectively suppressed between +0.2 and −0.2 V. Other studies used this electro-fermentation approach in pure culture systems to produce chemicals including ethanol, butanol, lactate, and lysine.

In one embodiment, the methods described herein use solid electrodes as an alternative source or sink of electrons to regulate the redox potential of the mixed culture reactor fed with glucose, which can therefore force the rebalance the fermentation pathways to promote or inhibit targeted processes. The product spectrum was systematically evaluated under a large range of redox conditions from −1.0 V to −0.2 V, which covers the corresponding electron flow direction change from cathodic current to anodic current. The effects on methanogenesis under different conditions were specifically evaluated, and process stability was monitored for more than 3 months. System performance was also evaluated under both neutral and acidic conditions with different buffer capacities to understand the interactions between redox and pH controls.

### Anodic and Cathodic Current Monitored for EF

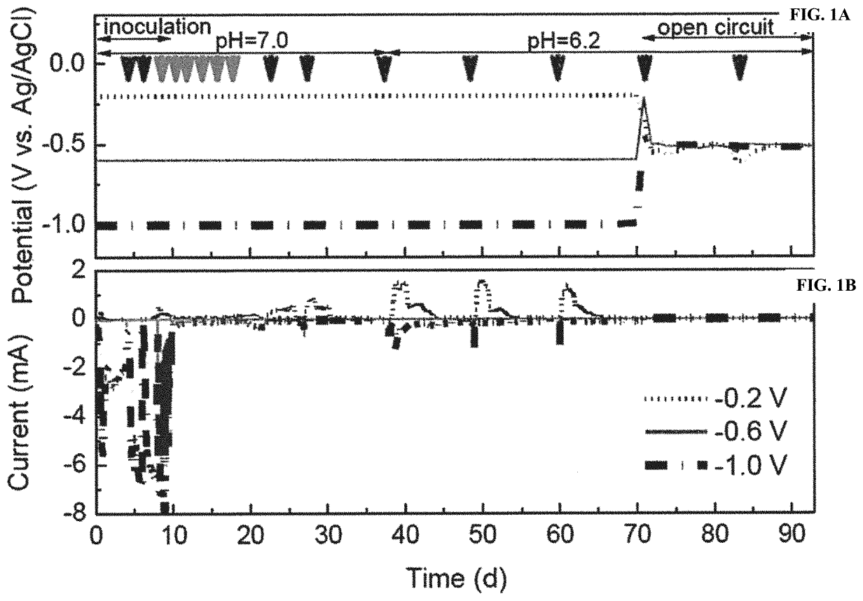

The EF reactors were operated in 2 stages. From day 1-70, the reactors were divided in 3 groups, with each group applied a fixed potential at −0.2 V, −0.6 V, and −1.0 V, respectively. From day 70-93, all reactors were operated in open circuit condition without poised potential, so the results can be used to reveal the effects of electric current on product selection (FIG. 1A). It can be seen that during the open circuit mode the working potentials of all reactors were approximately −0.55 V regardless of the original states. The feeding frequency for the fed-batch operation extended from 2 days to 5 days and finally to 11 days to achieve a steady state, as shown with yellow arrows in FIG. 1A. FIG. 1B shows that when a working potential of −0.2 V was applied, the electrode potential was higher than the open circuit potential (−0.55 V), so anodic EF reactions occurred with electrons flowing toward the electrode, showing in positive current. In contrast, when the applied potentials were lower than the open circuit potential (−0.6 V, −1.0 V), cathodic EF conditions were created, where the working electrode became an electron source, showing in negative current. The current at −1.0 V showed significant fluctuation at the beginning before stabilizing after 10 days, while the currents in the other two conditions were relatively stable. The maximum current in each batch was observed right after each substrate replacement, indicating possible disturbance of the redox environment. The maximum anodic current was 1.47±0.12 mA, while the maximum cathodic current was −0.14±0.03 mA during stable operation.

Without being bound by theory, it is believed that in mixed culture fermentation reactors, fermentative bacteria and electroactive bacteria interact synergistically to sustain the conversion of organic substrates to currents in an anode EF condition, while in cathode EF condition, the microbial consortium consumes electrons from the electrode to make different products depending on the redox conditions.

### Applied Working Potentials Affected Hydrogen and Methane Production

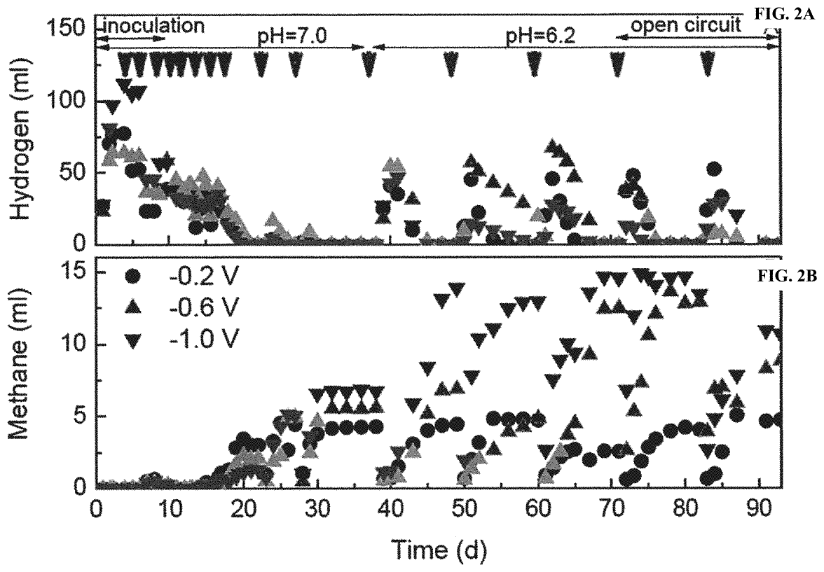

FIG. 2 shows the profiles of H₂ and CH₄ generation during the operation in different stages. All reactors showed fast H₂ generation at the beginning, presumably due to fermentation of easily degradable glucose. Higher H₂ was produced during the stage when initial electrolyte pH was maintained at 7.0, which is correlated with higher acetic acid and butyric acid production that will be discussed later. Without being bound by theory, it is believed that frequent substrate change at the beginning of the experiment did not allow a clear result separation between batches as there were still significant amount of unused substrate remain. Without being bound by theory, it is believed that because H₂ was constantly consumed by hydrogenotrophic methanogens and electroactive bacteria, the observed volume was a combination of generation and consumption. When the duration of each batch extended to 5 days, a clear profile of decline in H₂ generation was observed for each batch. It should be noted that the H₂ production may not be likely from water electrolysis, as the water electrolysis requires a large cathodic current density as the sole source of electrons for protons reduction, while a consistently decrease of cathodic current density was observed here (FIG. 1B).

Interestingly, based at least in part on FIG. 2B, CH₄ generation appears to be significantly influenced by the poised potential in different EF reactors. Without being bound by theory, it is believed that that lower potential led to higher CH₄ accumulation, while in other words higher potential inhibited methanogenesis. For example, reactors under −1.0 V working potential consistently showed higher CH₄ generation than reactors under −0.6 V, which in turn showed higher CH₄ than reactors under −0.2 V. Overall, an average of 12.02±3.59 mL of CH₄ was generation from reactors under −1.0 V, where 7.44±3.46 mL and 3.99±0.98 mL were generation from reactors under −0.6 V and −0.2 V, respectively. This trend does not necessarily align with the H₂ content in each reactor, suggesting that the redox potential applied in each reactor played an important role in influencing methanogenesis. In certain embodiments, regulating the redox potential can be regulated using an electrode, methanogenesis can be effectively inhibited or improved depending on operational goals.

When all reactors were switched to open circuit, lower CH₄ production was observed in reactors previously operated at −0.6 and −1.0 V. In contrast, slightly higher CH₄ generation was observed in reactors operated at −0.2 V. Gradually all reactors started to show similar performance in gas production. Such convergence is understandable as reactors are now operated in the same condition, but it further confirms that electro-fermentation process can be significantly influenced by the redox potential applied on the reactor.

### Applied Working Potentials Affected Carboxylic Acids Production

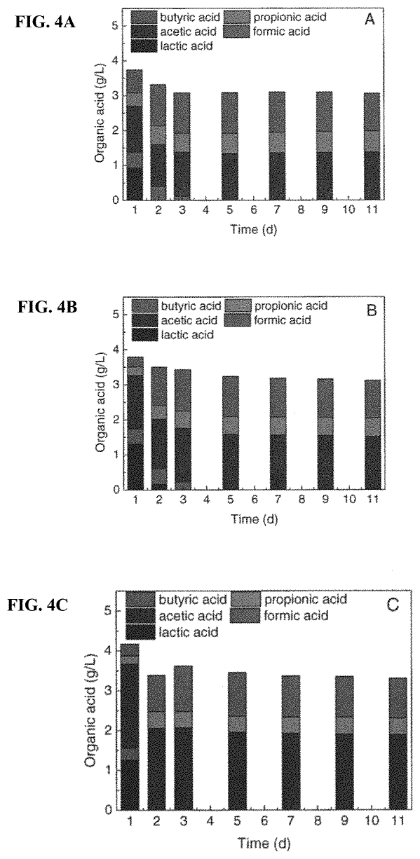

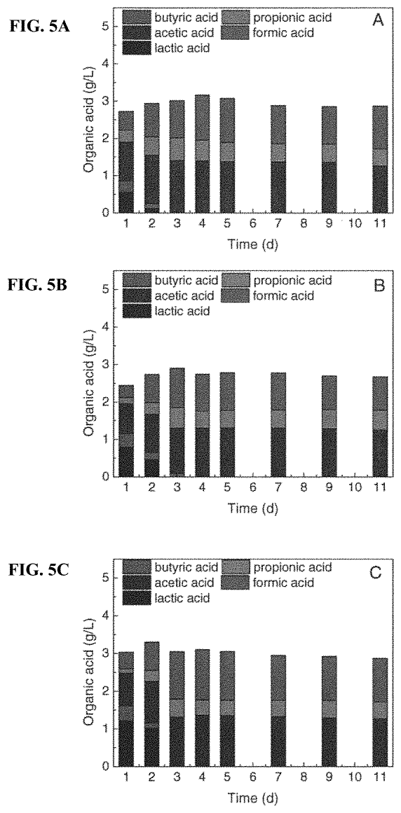

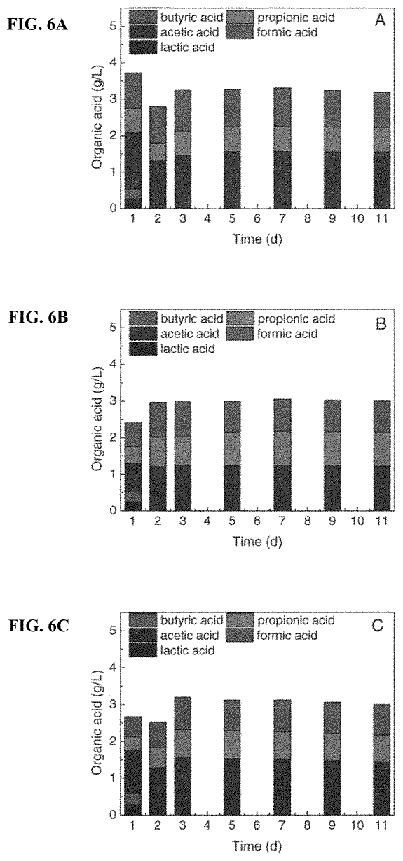

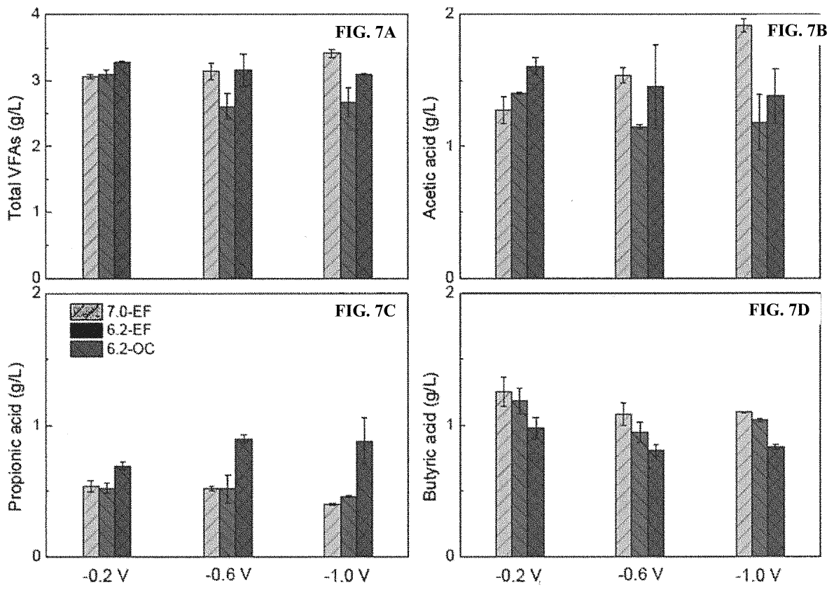

The production of different carboxylic acids including formic acid, acetic acid, propionic acid, butyric acid and lactic acid was measured under different working potentials in 3 stages. From day 0 to 37 days the electrolyte had a pH of 7.0, and then from day 38 to 93 the pH was artificially reduced to 6.2. After that, all reactors were operated at a same open circuit condition. FIGS. 4A-4C, FIGS. 5A-5C, and FIGS. 6A-6C show that formic acid and lactic acid were produced at the beginning then disappeared after approximately 3 days, suggesting that they may have been intermediates and consumed by microbes. The concentration and ratio became stable after 5 days of a batch test, therefore the concentration of major VFAs (acetic acid, propionic acid, and butyric acid) were compared at this condition (FIGS. 7A-7D). Overall the total amount of VFA generation did not change significantly under different working potentials, but lower potential did lead to slight increase in total VFA under pH 7.0 (FIG. 7A). Such increase is mainly attributed to the increase in acetic acid production. FIG. 7B shows that when the working potential dropped from −0.2 V to −1.0 Vat pH 7.0, the production of acetic acid increased by 55%. However, trend is not obvious in acidic condition (pH 6.2). The working potential did not seem to have big impact on propionic acid production, rather higher production was observed in open circuit potential (FIG. 7C). Interestingly, the generation of butyric acid showed an opposite trend compared to acetic acid, as reducing working potential led to a decrease in production in both pH conditions. Neutral pH did lead to higher butyric production across the different working potentials (FIG. 7D). Overall, the decrease of working potential promoted acetic acid production, showed little impacts on propionic acid, while slightly decreased butyric acid production. Plus, more propionic acids and less butyric acids production were observed when the reactors were switched from the controlled working potentials open circuit condition (FIG. 7C and FIG. 7D).

Microorganisms use a set of metabolic regulatory enzymes to sense the environmental redox state such as the presence of external electron sink, and such changes can trigger the shift of metabolic pathways. Without being bound by theory, it is believed that the working electrode introduced by EF can be regarded as a special external electron sink or source depending on the potential applied, which triggered the activities of regulatory enzymes. Specifically, the anodic EF of methods described herein is believed to drive more ATP synthesis by creating a proton gradient, while the electron supply by cathodic EF are expected to produce more reduced redox factors (NADPH). The carboxylic acids data are in agreement with the findings that less H₂ was produced when switched open circuit, as the H₂ production is only associated with acetic and butyric acids pathways. These pathways are regarded as major routes in anaerobic fermentation for ATP generation and have been found dominant in neutral pH condition.

### Analysis of Electroactivity of Working Electrodes.

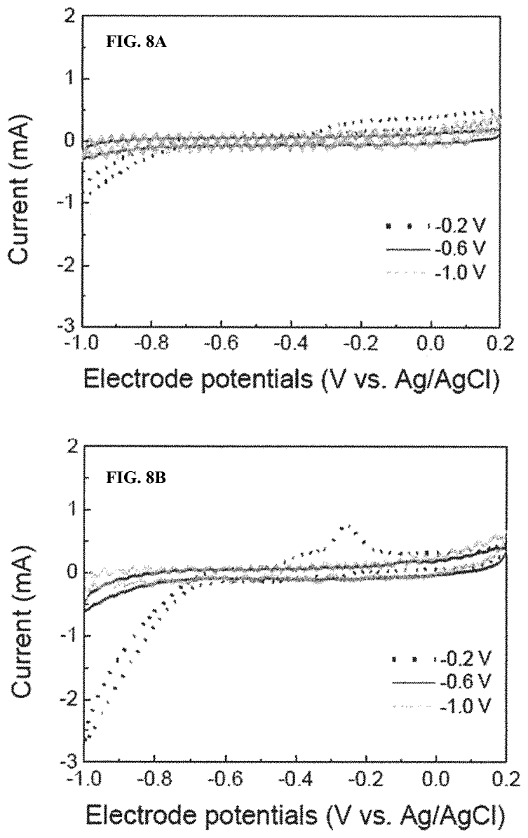

The electrochemical activities of the working electrodes under different working potentials were characterized using cyclic voltammetry (CV). FIG. 8A shows that no apparent redox peaks were observed in any reactors at the beginning of a typical batch cycle. However, by the end of the batch cycle, an oxidation peak at approximately −0.26 V was observed for reactor under −0.2 V, but no correlated reduction peak was detected (FIG. 8B). Without being bound by theory, it is believed that the hidden of reduction peak probably due to residue of unused substrates at the end of batch cycles, which make it more like a turnover CV condition. Without being bound by theory, it is believed that the oxidation peaks observed here can be associated with redox couples such as flavoproteins and/or iron-sulfur proteins. With regards to cathodic EF with potentials controlled at −0.6 V or −1.0 V, no redox peaks were found in CV curves. Without being bound by theory, it is believed that these CV results suggest that metabolites with electroactivity were generated during the anodic EF process, but the electron exchanges between microbes and the electrode in cathodic EF did not involve mediated process.

### Methane Production Kinetics of the Anaerobic Sludge Used as Inoculum

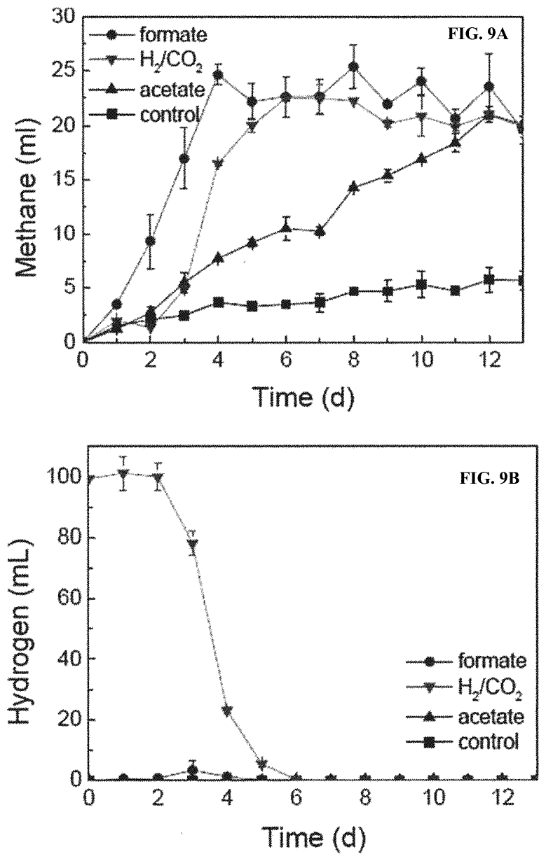

To further understand CH₄ generation potential and kinetics, serum bottle tests were conducted. Each bottle contained a 50-50 mixture of the sludge inoculum and a pure substrate for each known group of methanogens (formate, acetate, or H₂/CO₂) For all reactors, CH₄ generation increased with time until a plateau was reached, which is considered as the CH₄ generation potential (FIG. 9A). However, the CH₄ production rate was significantly different from different mixtures. The formate-fed reactor showed the highest production rate (115±5 mL/L/day), which was followed by the H₂/CO₂ reactor (75±2 mL/L/day). The acetate amended reactor showed the slowest response (35±1 mL/L/day). The CH₄ production rate was further confirmed by the H₂ consumption rate, where a lag phase was observed when fed with H₂/CO₂ (FIG. 9B). The CH₄ production rates were similar as previous studies using sludge from a granular sludge system. These results help better explain the findings in EF reactors, in which H₂ was quickly consumed but acetate remained abundant, suggesting hydrogenotrophic methanogen played a major role in CH₄ production than acetoclastic methanogens. This is beneficial to the EF system as VFAs are desired products over gaseous products. FIGS. 4A-4C, FIGS. 5A-5C, and FIGS. 6A-6C show that formate was formed at the beginning in small amount but quickly disappeared, suggesting it was an intermediate product that was not significant related to methanogenesis. This is further confirmed by the observation that three types of VFAs, acetic acid, propionic acid, and butyric acid were the major end products after EF operation.

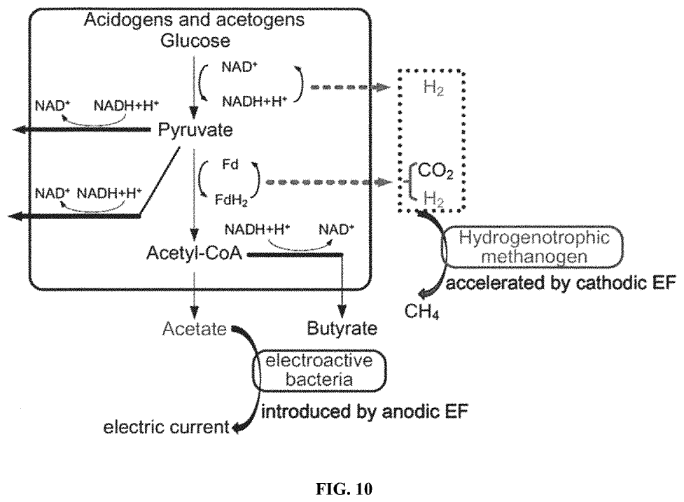

The results indicate that electrical potential had direct impacts on mixed culture fermentation and influenced metabolites generation. Unlike traditional anaerobic digestion in which biogas is the main product after organic degradation, and without being bound by theory, it is believed that EF reactors showed various distribution of final products depending on the applied electrical potential. Low working potential was associated with cathodic current, which promoted the production of acetic acid and H₂ and boosted methanogenesis (FIG. 10). In contrast, higher working potential was associated with anodic current, which inhibited methanogen growth, rather promoted the growth of electroactive bacteria and accumulation of butyric acid.

### EXAMPLES

Various embodiments of the present application can be better understood by reference to the following Examples which are offered by way of illustration. The scope of the present application is not limited to the Examples given herein.

### Example 1: EF Reactors Construction and Startup

Each EF reactor had two chambers which were separated with a cation exchange membrane (CMI7000, Membranes International Inc., NJ, USA). Each working electrode chamber had a volume of 100 mL, and each counter electrode chamber had a volume of 25 mL. Graphite felts (Sanye Carbon Co., Ltd., Beijing, China) were used as electrodes. Each electrode was in disc shape with a projected area of 28 cm² A gas bag was connected to the working electrode chamber using a collection tube. Each reactor was equipped with an Ag/AgCl reference electrode (3 M KCl, AgCl saturated, +0.2 V versus SHE) placed near the working electrode. All potentials reported in the study are relative to the Ag/AgCl reference electrode.

In one embodiment, the first electrode includes neutral red covalently bound to graphite felt. It has been surprisingly discovered that the efficiency of electron transfer from microbial cells to electrodes can be increased by covalently linking neutral red to woven graphite felt. Neutral red can be covalently linked to graphite felt by converting at least a portion of the surface of the graphite felt to its carboxy form (using heat, for example) and covalently linking the amine group of neutral red to the carboxy of the woven graphite. The neutral red is immobilized on the graphite felt and does not leach-out in water.

The first electrode comprising neutral red covalently bound to graphite felt may then be incorporated into an electrochemical bioreactor system using known methods within the ability of one skilled in the art. A typical electrochemical bioreactor system will include two compartments, each of which contains one of the first and the second electrodes. The second electrode can comprise an iron cation (e.g., Fe³⁺) associated with a graphite plate. The electrodes are separated by an ionically conductive electrolyte material between the first electrode and the second electrode. Typically, the electrolyte allows for the passage of protons and cations only. Solid electrolyte materials are well known and include materials such as Nafion™ cationic selective membranes and porcelain septums. Catholytes and anolytes can also be used in the compartments. For example, catholytes and anolytes that have been found to be suitable in electrochemical bioreactors include bacterial growth media or a phosphate buffer. The electrodes can be connected to an electrical power source or to a multimeter (or other resistive load) using an electrically conductive material such as a metallic material (e.g., platinum wire).

A suitable biocatalyst is disposed in the compartment containing the first electrode or is associated (as defined above) with the first electrode. The membrane permeable to protons can be a proton exchange membrane. Glucose based synthetic wastewater was used as the substrate in all reactors. Every liter of growth medium contained 5 g glucose, 0.5 g NH₄Cl, 0.5 g KCl, 0.1 g CaCl₂, 0.1 g MgCl₂.6H₂O, and 10 mL trace minerals solution, and 200 mM PBS solution (with the initial pH of 7.0 or 6.2 as described in following section). The growth medium was purged with N₂ prior to feeding to achieve an anaerobic condition. The same PBS solution without substrate was used as the electrolyte in the abiotic counter electrode chamber. Anaerobic sludge taken from a local wastewater treatment plant (Boulder, Colo.) was used as the inoculum (10% v/v in the initial 3 batches). The reactors were operated in fed-batch mode in different conditions for a total period of 93 days. The operation temperature was controlled at 35° C., and the solution was constantly mixed by a magnetic stir bar. The working potentials were controlled using a multichannel potentiostat (CHI 1000B, Chenhua Co., Shanghai, China) in the three-electrode mode, and three potentials of −0.2 V, −0.6 V, and −1.0 V were applied in different reactors, respectively.

### Example 2: Biocatalysts to Promote Cell Growth or the Formation of Reduced Products in the Electrochemical Bioreactor System

A wide range of biocatalysts to promote cell growth or the formation of reduced products in the electrochemical bioreactor system can be used. For example, a wide variety of bacteria, archea, plant cells or animal cells can be used. Non-limiting examples include cells of Actinobacillus succinogenes, cells of Escherichia coli, and sewage sludge. Enzyme preparations can also be used in the practice of the methods described herein. A desired enzyme can be partially purified using standard methods known to one of ordinary skill in the art. The enzyme can be isolated from its native source or from a transgenic expression host, or obtained through a commercial vendor. Useful enzymes include any enzyme that can use reducing power from an electron mediator to form a desired reduced product, or which can transfer reducing power to an electron mediator and form a desired oxidized product. Most commonly, this reduction is mediated by NADPH or NADH. An oxidoreductase can be used as the biocatalyst in the practice of the methods described herein. For example, isolated alcohol dehydrogenases, carboxylic acid reductase, and fumarate reductase could be used in the electrochemical bioreactor system.

In another embodiment, the first electrode of the electrochemical bioreactor system includes a metal ion electron mediator. For example, the first electrode may comprise an iron cation (e.g., Fe³+) and/or a manganese cation (e.g., Mn⁴⁺) associated with a graphite plate. The first electrode including the metal ion electron mediator can be incorporated into an electrochemical bioreactor system using the methods as described herein. The present methods also provide for utilizing oxidoreductase as biocatalysts for chemical sensing and chemical production, and as a biofuel cell by immobilizing on an electrode an oxidoreductase and multiple electron carriers such as nicotinamide adenine dinucleotide (NAD+) and neutral red (NR) which can be bioelectrically regenerated. In one embodiment, fumarate reductase enzyme is immobilized onto a graphite felt electrode that is modified with carboxymethylcellulose (CMC), neutral red (NR) and nicotinamide adenine dinucleotide (NAD+). The fumarate reductase enzyme is immobilized onto a CMC-NR-NAD+ modified graphite felt electrode by preparing fumarate reductase, preparing a graphite electrode substrate, treating the electrode with neutral red, treating the electrode with carboxymethylcellulose, treating the electrode with NAD+, and treating the electrode with the fumarate reductase to produce a CMC-NR-NAD+-fumarate reductase modified electrode. The membrane permeable to protons may be a proton exchange membrane.

The bioreactor can contain dissolved nutrients for facilitating growth of the iron-oxidizing microorganisms. Controlling a ratio of electrical production to biomass production can be achieved by varying microbial cultivation parameters including an electrical potential of the cathode electrode, by varying the inorganic nutrient salt composition, or a combination of these.

### Example 3: Analyses and Calculations

The current across each reactor was constantly monitored by the multichannel potentiostat. The concentration of carbohydrates including formic acid, acetic acid propionic acid, butyric acid and lactic acid was analyzed by HPLC (Agilent 1200, USA) using a Bio-Rad Aminex HPX-87C column with 10 mM phosphoric acid solution as the eluent at a flow rate of 1 mL min⁻¹. The concentration and volume of hydrogen and methane were quantified by a gas chromatograph (Model 8610C, SRI Instruments) equipped with a thermal conductivity detector using nitrogen as the carrier gas. The electrochemical characteristics of the working electrode was analyzed by cyclic voltammetry (CV) from −1.0 V to +0.2 V at a scan rate of 1 mV s⁻¹.

The methane production kinetics of the anaerobic sludge inoculum for electro-fermentation were investigated using 250 mL serum bottles with a working volume of 5 mL. Each bottle contained 25 mL of anaerobic sludge and 25 mL medium used in the EF reactors but without glucose. Duplicate reactors were used, with the control containing no additional electron donors. In addition, the medium was supplemented with a mixture of formate (4 mmol), acetate (1 mmol), and H₂/CO₂ (80:20 v/v, 5 mmol), respectively, with the goal of obtaining the same theoretical CH₄ potential (1 mmol) based on stoichiometry.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a method of producing the fermentation products of mixed culture, the method comprising:
a) providing:

i) a mixed culture comprising at least two different types of microorganisms;

ii) at least one reactor, wherein said reactor comprises at least one chamber, wherein the at least one chamber comprises the mixed culture, at least one anode, and at least one cathode, and wherein the reactor has an open circuit potential; and

iii) optionally an ion exchange membrane or other separator in the at least one chamber;
b) adjusting the electrical potential on the at least one anode and at least one cathode to regulate the fermentation products of said mixed culture; and
c) collecting the fermentation products.

Embodiment 2 provides the method of embodiment 1, wherein the reactor comprises a first chamber and a second chamber.

Embodiment 3 provides the method of any one of embodiments 1-2, wherein the first chamber comprises at least one anode and the second chamber comprises at least one cathode.

Embodiment 4 provides the method of any one of embodiments 1-3, wherein the ion exchange membrane or other separator separates the first and second chamber.

Embodiment 5 provides the method of any one of embodiments 1-4, wherein said reactor comprises an anaerobic bioreactor.

Embodiment 6 provides the method of any one of embodiments 1-5, wherein said fermentation products comprise at least one of a short chain volatile fatty acid, an alcohol, hydrogen, or methane gas.

Embodiment 7 provides the method of any one of embodiments 1-6, wherein a reactor working potential less than the open circuit potential induces a cathodic current and increases CH₄ generation.

Embodiment 8 provides the method of any one of embodiments 1-7, wherein said reactor working potential is less than −0.2 V vs. Ag/AgCl.

Embodiment 9 provides the method of any one of embodiments 1-8, wherein a reactor working potential greater than the open circuit potential induces an anodic current and increases short chain volatile fatty acid generation.

Embodiment 10 provides the method of any one of embodiments 1-9, wherein said reactor working potential is greater than −0.6 V vs. Ag/AgCl.

Embodiment 11 provides the method of any one of embodiments 1-10, wherein said applied potentials on the electrodes influence the fermentation product distribution.

Embodiment 12 provides the method of any one of embodiments 1-11, wherein said reactor further comprises a Ag/AgCl reference electrode.

Embodiment 13 provides the method of any one of embodiments 1-12, wherein the mixed culture comprises at least one organism selected from the group consisting of Actinobacillus succinogenes, Escherichia coli, M. thermautotrophicus, fermentative bacteria, methanogens, acetogenic bacteria, acidogenic bacteria, and electroactive bacteria.

Embodiment 14 provides the method of any one of embodiments 1-13, wherein the mixed culture further comprises anaerobic wastewater sludge, food waste, animal waste, wastewater, or other wet organic wastes.

Embodiment 15 provides the method of any one of embodiments 1-14, wherein the short chain volatile fatty acid comprises at least one short chain fatty acid chosen from acetic acid, propionic acid, butyric acid, or lactic acid.

Embodiment 16 provides a method of producing the fermentation products of mixed culture, the method comprising:
a) providing:
   i) a mixed culture comprising at least two different types of microorganisms;
   ii) at least one reactor, wherein said reactor comprises at least one chamber, wherein the at least one chamber comprises the mixed culture, at least one anode, and at least one cathode, and wherein the reactor has an open circuit potential; and
   iii) optionally an ion exchange membrane or other separator in the at least one chamber;
b) adjusting the electrical potential on the at least one anode and at least one cathode to reduce methanogensis in the mixed culture by at least 20%.

Embodiment 17 provides the method of embodiment 16, wherein the method further comprises changing butyric acid production in the mixed culture by at least 25%.

Embodiment 18 provides the method of any one of embodiments 16-17, wherein the method further comprises changing acetic acid generation in the mixed culture by at least 25%.

Embodiment 19 provides the method of any one of embodiments 16-18, wherein the reactor comprises a first chamber and a second chamber.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of producing the fermentation products of mixed culture, the method comprising:
a) providing:
i) a mixed culture comprising at least two different types of microorganisms;
ii) at least one reactor, wherein said reactor comprises at least one chamber, wherein the at least one chamber comprises the mixed culture, at least one anode, and at least one cathode, and wherein the reactor has an open circuit potential; and
iii) optionally an ion exchange membrane or other separator in the at least one chamber;
b) adjusting the electrical potential on the at least one anode and at least one cathode to regulate the fermentation products of said mixed culture; and
c) collecting the fermentation products.

2. The method of claim 1, wherein the reactor comprises a first chamber and a second chamber.

3. The method of claim 2, wherein the first chamber comprises at least one anode and the second chamber comprises at least one cathode.

4. The method of claim 3, wherein the ion exchange membrane or other separator separates the first and second chamber.

5. The method of claim 1, wherein said reactor comprises an anaerobic bioreactor.

6. The method of claim 1, wherein said fermentation products comprise at least one of a short chain volatile fatty acid, an alcohol, hydrogen, or methane gas.

7. The method of claim 1, wherein a reactor working potential less than the open circuit potential induces a cathodic current and increases CH₄ generation.

8. The method of claim 7, wherein said reactor working potential is less than −0.2 V vs. Ag/AgCl.

9. The method of claim 1, wherein a reactor working potential greater than the open circuit potential induces an anodic current and increases short chain volatile fatty acid generation.

10. The method of claim 9, wherein said reactor working potential is greater than −0.6 V vs. Ag/AgCl.

11. The method of claim 1, wherein said applied potentials on the electrodes influence the fermentation product distribution.

12. The method of claim 1, wherein said reactor further comprises a Ag/AgCl reference electrode.

13. The method of claim 1, wherein the mixed culture comprises at least one organism selected from the group consisting of fermentative bacteria, methanogens, acetogenic bacteria, acidogenic bacteria, and electroactive bacteria.

14. The method of claim 13, wherein the mixed culture further comprises anaerobic wastewater sludge, food waste, animal waste, wastewater, or other wet organic wastes.

15. The method of claim 6, wherein the short chain volatile fatty acid comprises at least one short chain fatty acid chosen from acetic acid, propionic acid, butyric acid, or lactic acid.

16. A method of producing the fermentation products of mixed culture, the method comprising:
a) providing:
i) a mixed culture comprising at least two different types of microorganisms;
ii) at least one reactor, wherein said reactor comprises at least one chamber, wherein the at least one chamber comprises the mixed culture, at least one anode, and at least one cathode, and wherein the reactor has an open circuit potential; and
iii) optionally an ion exchange membrane or other separator in the at least one chamber; and
b) adjusting the electrical potential on the at least one anode and at least one cathode to reduce methanogensis in the mixed culture by at least 20%.

17. The method of claim 16, wherein the method further comprises changing butyric acid production in the mixed culture by at least 25%.

18. The method of claim 16, wherein the method further comprises changing acetic acid generation in the mixed culture by at least 25%.

19. The method of claim 16, wherein the reactor comprises a first chamber and a second chamber.
